(19) United States
(12) Patent Application Publication
(10) Pub. No.: US 2019/0208985 A1
(43) Pub. Date: Jul. 11, 2019

(54) ELECTRONIC ENDOSCOPE SYSTEM

(71) Applicant: HOYA CORPORATION, Tokyo (JP)

(72) Inventor: Masaaki FUKUDA, Tokyo (JP)

(73) Assignee: HOYA CORPORATION, Tokyo (JP)

(21) Appl. No.: 16/315,804

(22) PCT Filed: Sep. 1, 2017
(86) PCT No.: PCT/JP2017/031669

§ 371 Date: Jan. 7, 2019

(30) Foreign Application Priority Data
Sep. 2, 2016 (JP) 2016-171586

(51) Int. Cl. A61B 1/00; A61B 1/045; A61B 1/07

(52) U.S. Cl. CPC A61B 1/00009; A61B 1/045; A61B 5/1459; A61B 1/00186; A61B 1/07

(57) ABSTRACT

Provided is an electronic endoscope system including: image signal generation means for sequentially taking images of a subject sequentially irradiated with multiple types of irradiation light having different spectrums and generating image signals of the subject irradiated with the respective types of irradiation light as image signals of multiple systems; storage means having a predetermined correction value stored therein in advance; and spectral image generation means for generating a spectral image based on image signals of at least two systems among the image signals of multiple systems generated by the image signal generation means. When generating the spectral image based on the image signals of the at least two systems, the spectral image generation means corrects an image signal of at least one system among the image signals of the at least two systems based on the correction value stored in advance in the storage means.

### CROSS-REFERENCE TO RELATED APPLICATIONS

This application is a 371 U.S. National Phase of PCT International Application No. PCT/JP2017/031669, filed on Sep. 1, 2017, which claims benefit and priority to Japanese patent application No. 2016-171586, filed on Sep. 2, 2016, and which is incorporated by reference herein in its entirety.

### TECHNICAL FIELD

The present disclosure relates to an electronic endoscope system.

### BACKGROUND ART

Endoscope systems capable of taking special images are known. For example, Patent Document 1 discloses a specific configuration of an endoscope system of this type.

The endoscope system disclosed in Patent Document 1 includes a light source device. The light source device disclosed in Patent Document 1 has a rotary filter mounted thereon. In this rotary filter, three optical bandpass filters (two optical bandpass filters for selectively transmitting 550 nm-band light and one optical bandpass filter for selectively transmitting 650 nm-band light) and a normal observation filter for transmitting white light are arranged side by side in the circumferential direction. A controller drives the rotary filter to rotate at a fixed rotation cycle, sequentially inserts the filters into the optical path of white light, and sequentially takes images of biological tissue by using irradiation light passing through the filters. The controller generates an image (for example, an image indicating the distribution of hemoglobin oxygen saturation) indicating the distribution of biomolecules in the biological tissue based on data of images taken by using the respective optical bandpass filters, and arranges the generated distribution image side by side with a normal observation image taken by using the normal observation filter to be displayed within a display screen.

### CITATION LIST

### Patent Document

Patent Document 1: WO 2014/192781 A

### SUMMARY OF INVENTION

### Technical Problem

In Patent Document 1, when the electronic endoscope system has an individual difference (for example, an individual difference in terms of the spectral characteristics of the optical bandpass filters, the sensitivity of the solid-state imaging device, or the like), error will be included the results of calculation of oxygen saturation, etc., which is performed based on image data that was taken. A problem can be pointed out that when there is a large individual difference of such a type, it is difficult to generate spectral images having high accuracy.

The present disclosure has been made in view of the above-described circumstances, and an aim thereof is to provide an electronic endoscope system that is suitable for suppressing a degradation, deriving from an individual difference of the system, in the accuracy of calculation of information such as oxygen saturation that is necessary for the generation of a spectral image.

### Solution to the Problem

An electronic endoscope system pertaining to one embodiment of the present disclosure includes: an irradiation means for sequentially irradiating a subject with multiple types of irradiation light having different spectrums; an image signal generation means for sequentially taking images of the subject sequentially irradiated with the multiple types of irradiation light and generating image signals of the subject irradiated with the respective types of irradiation light as image signals of multiple systems; a storage means having a predetermined correction value stored therein in advance; and a spectral image generation means for generating a spectral image based on image signals of at least two systems among the image signals of multiple systems generated by the image signal generation means. When generating the spectral image of a feature amount of the subject determinable based on the image signals of the at least two systems, the spectral image generation means corrects an image signal of at least one system among the image signals of the at least two systems based on the correction value stored in advance in the storage means.

Furthermore, according to one embodiment of the present disclosure, the correction value is a value calculated in advance based on a ratio between luminance values of a specific pair of image signals among the image signals of the at least two systems. In this case, when generating the spectral image based on the image signals of the at least two systems, the spectral image generation means preferably corrects one image signal among the specific pair of image signals based on the correction value.

Furthermore, according to one embodiment of the present disclosure, the correction value preferably is a correction value set so that a ratio between luminance values of the specific pair of image signals equals a predetermined target ratio, the luminance values of the specific pair of image signals being yielded when images are taken of a reference subject irradiated with the multiple types of irradiation light.

Furthermore, according to one embodiment of the present disclosure, the irradiation means preferably includes: a light source for emitting light; a rotary member in which a plurality of light transmission regions having different transmission bands are arranged side by side in a circumferential direction; a means for causing the rotary member to rotate and sequentially inserting the plurality of light transmission regions into an optical path of the light in order to sequentially take out the multiple types of irradiation light having different spectrums from the light emitted from the light source; and a means for sequentially emitting, toward the subject, the multiple types of irradiation light that are sequentially taken out.

Furthermore, according to one embodiment of the present disclosure, it is preferable that the plurality of light transmission regions are optical filters that are arranged in the rotary member, the optical filters including: a first filter having a first transmission band, the first transmission band being included within a wavelength band of 520 to 590; a second filter having a second transmission band, the second transmission band being included within the wavelength band of 520 to 590 and being narrower than the first transmission band; and a filter that transmits white light.

Furthermore, according to one embodiment of the present disclosure, the correction value stored in advance in the storage means includes a first correction value. The first correction value preferably is a value for correcting, into a predetermined first ratio, a ratio between a luminance value of an image signal constituted of a part of a plurality of components constituting an image signal of the reference subject irradiated with the white light and a luminance value of an image signal of the reference subject irradiated with light filtered by the first filter. In this case, the spectral image generation means preferably corrects an image signal A constituted of a part of a plurality of components constituting an image signal of the subject irradiated with the white light based on the first correction value, divides an image signal B of the subject irradiated with light filtered by the first filter by the image signal A corrected with the first correction value to acquire hemoglobin concentration information of the subject, and generates a spectral image indicating hemoglobin concentration based on the acquired hemoglobin concentration information.

Furthermore, according to one embodiment of the present disclosure, the correction value stored in advance in the storage means includes a second correction value. For example, the second correction value preferably is a value for correcting, into a predetermined second ratio, a ratio between the luminance value of the image signal of the reference subject irradiated with the light filtered by the first filter and a luminance value of an image signal of the reference subject irradiated with light filtered by the second filter. In this case, the spectral image generation means preferably corrects the image signal A constituted of a part of the plurality of components constituting the image signal of the subject irradiated with the white light based on the first correction value, and also corrects an image signal C of the subject irradiated with light filtered by the second filter based on the second correction value, subtracts the image signal C corrected using the second correction value from the image signal B of the subject irradiated with the light filtered by the first filter, divides the value after the subtraction by the image signal A corrected using the first correction value to acquire oxygen saturation information of the subject, and generates a spectral image indicating oxygen saturation based on the acquired oxygen saturation information

An electronic endoscope system pertaining to another embodiment of the present disclosure includes: an irradiation means for sequentially irradiating a subject with multiple types of irradiation light having different spectrums; an image signal generation means for sequentially taking images of the subject sequentially irradiated with the multiple types of irradiation light and generating image signals of the subject irradiated with the respective types of irradiation light as image signals of multiple systems; and a spectral image generation means for generating a spectral image indicating a distribution of a feature amount of the subject, the feature amount being determinable based on image signals of at least two systems among the image signals of multiple systems.

The spectral image generation means calculates the feature amount by correcting one of the image signals of the at least two systems based on a predetermined correction value, and the predetermined correction value is a correction value set so that a ratio between luminance values of reference image signals of the at least two systems equals a predetermined target ratio, the reference image signals of the at least two systems being yielded when images are taken of a reference subject irradiated with the irradiation light.

Furthermore, according to one embodiment of the present disclosure, the feature amount preferably is an amount determinable based on a ratio between luminance values of the image signals of the at least two systems.

Furthermore, according to one embodiment of the present disclosure, a wavelength band of one type of irradiation light, among the multiple types of irradiation light, preferably is demarcated from a wavelength band of another type of irradiation light, among the multiple types of irradiation light, by an isosbestic point corresponding to a switch between levels of a spectral waveform of light absorbance of oxygenated hemoglobin and a spectral waveform of light absorbance of reduced hemoglobin.

According to one embodiment of the present disclosure, a wavelength band of one type of irradiation light, among the multiple types of irradiation light, preferably is included within a wavelength band between isosbestic points that are adjacent in a wavelength direction, among a plurality of isosbestic points corresponding to a switch between levels of the spectral waveform of light absorbance of oxygenated hemoglobin and the spectral waveform of light absorbance of reduced hemoglobin.

Note that, according to the later-described embodiment illustrated in FIG. 1, the above-described irradiation means preferably includes a light source device including: a lamp 208; a rotary filter portion 260; and a light condenser lens 210. Furthermore, it is also preferable that the irradiation means is configured to have a plurality of light-emitting diodes for emitting multiple types of irradiation light, and to sequentially emit multiple types of light.

According to the later-described embodiment illustrated in FIG. 1, the above-described image signal generation means preferably includes a driver signal processing circuit 110.

According to the later-described embodiment illustrated in FIG. 4, the above-described storage means preferably includes a correction value memory 220F.

According to the later-described embodiment illustrated in FIG. 4, the above-described spectral image generation means preferably includes: a hemoglobin concentration calculation circuit 220D; an oxygen saturation calculation circuit 220E; and an image processing circuit 220B.

According to the later-described embodiment illustrated in FIG. 1, the above-described means for sequentially inserting the light transmission regions into the optical path of light preferably includes: a DC motor 262; and a rotary turret 261.

### Advantageous Effects of the Invention

According to the above-described endoscope system, it is possible to suppress a degradation, deriving from an individual difference of the system, in the accuracy of calculation of information of a feature amount such as oxygen saturation that is necessary for the generation of a spectral image.

### BRIEF DESCRIPTION OF DRAWINGS

FIG. 1 is a block diagram illustrating a configuration of an electronic endoscope system pertaining to one embodiment of the present disclosure.

FIG. 2 is a front view of a rotary filter portion included in a processor pertaining to one embodiment of the present disclosure, when seen from a light condenser lens side.

FIG. 3 is diagram illustrating a hemoglobin absorption spectrum, in which the vicinity of a 550 nm wavelength is illustrated in an enlarged state.

FIG. 4 is a block diagram illustrating a configuration of a signal processing circuit included in the processor pertaining to one embodiment of the present disclosure.

### DESCRIPTION OF EMBODIMENTS

In the following, an embodiment of the present disclosure is described with reference to the drawings. Note that in the following, description is provided taking an electronic endoscope system as an example of one embodiment of the present disclosure. The electronic endoscope system pertaining to the present embodiment is a system that is capable of quantitatively analyzing a feature amount of biological tissue, such as biological information (e.g., oxygen saturation, hemoglobin concentration), based on a plurality of images taken by using light having different spectrums, and is capable of presenting the feature amount as an image.

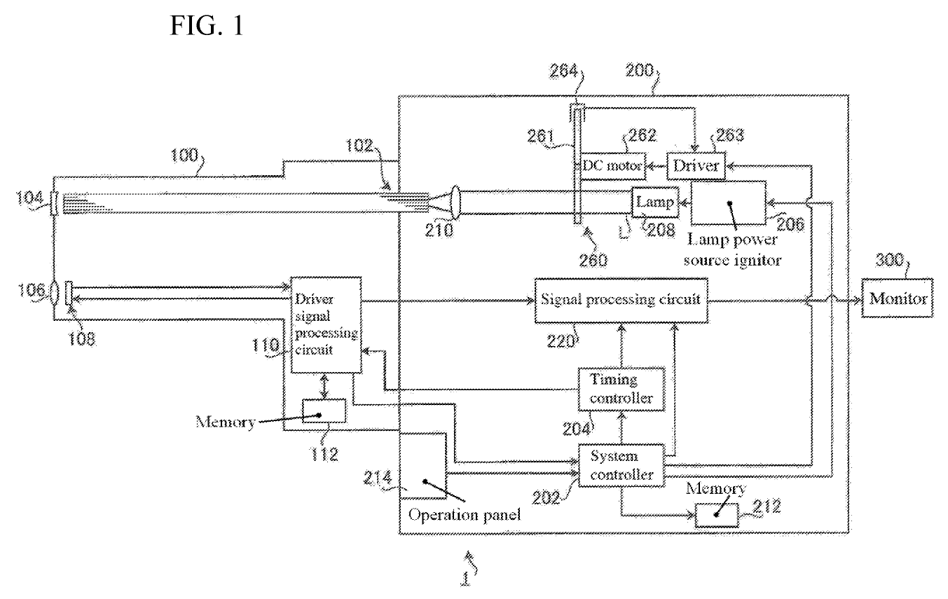

FIG. 1 is a block diagram illustrating a configuration of an electronic endoscope system 1 pertaining to one embodiment of the present disclosure. As illustrated in FIG. 1, the electronic endoscope system 1 pertaining to the present embodiment includes an electronic scope 100, a processor 200, and a monitor 300.

The processor 200 includes a system controller 202 and a timing controller 204. The system controller 202 executes various programs stored in a memory 212 and generally controls the entire electronic endoscope system 1. Furthermore, the system controller 202 is connected to an operation panel 214. In accordance with instructions from an operator input from the operation panel 214, the system controller 202 changes the operations of the electronic endoscope system 1 and the parameters for the operations. The timing controller 204 outputs, to circuits inside the electronic endoscope system 1, a clock pulse for adjusting the operation timing of various portions.

A lamp 208 emits irradiation light L after being started by a lamp power source ignitor 206. For example, the lamp 208 is a high intensity lamp such as a xenon lamp, a halogen lamp, a mercury lamp, a metal halide lamp, or the like, or a light-emitting diode (LED). The irradiation light L is light having a spectrum mainly spreading from the visible light region to the invisible infrared light region (or white light including at least the visible light region).

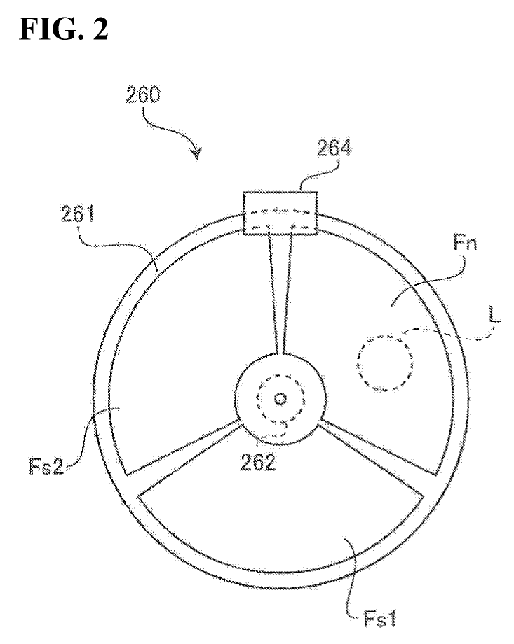

The irradiation light L emitted from the lamp 208 is made incident upon a rotary filter portion 260. FIG. 2 is a front view of the rotary filter portion 260 when seen from a light condenser lens 210 side. As illustrated in FIGS. 1 and 2, the rotary filter portion 260 includes a rotary turret 261, a DC motor 262, a driver 263, and a photo-interrupter 264.

As illustrated in FIG. 2, three optical filters are arranged in the rotary turret 261. Specifically, in the rotary turret 261, a normal observation (white light) filter Fn, a first special observation filter Fs1, and a second special observation filter Fs2 are arranged in order side by side in the circumferential direction. The optical filters have fan shapes extending over substantially the same angular range and are arranged at a 120° angular pitch.

The optical filters in the rotary turret 261 are all multilayered dielectric filters. However, optical filters of other forms (for example, etalon filters in which dielectric multilayer films are used as reflection films, etc.) may be used.

Here, description is provided of the spectral characteristics of hemoglobin.

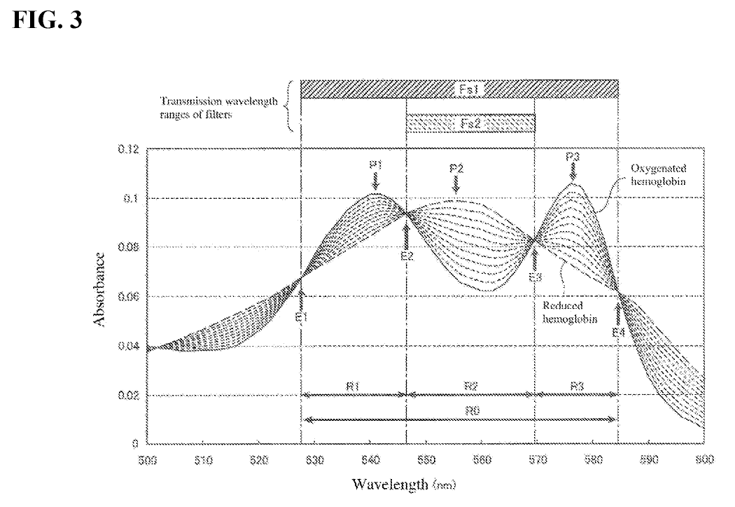

FIG. 3 illustrates the absorption spectrum of hemoglobin in the vicinity of the 550 nm wavelength. In the vicinity of the 550 nm wavelength, hemoglobin has a strong absorption band deriving from porphyrin, and this absorption band is referred to as the Q band. The absorption spectrum of hemoglobin varies depending upon oxygen saturation (the fraction of oxygenated hemoglobin relative to total hemoglobin). The waveform illustrated by using a solid line in FIG. 3 indicates the absorption spectrum when oxygen saturation is 100% (that is, the absorption spectrum of oxygenated hemoglobin HbO), and the waveform illustrated by using the long-dashed line in FIG. 3 indicates the absorption spectrum when oxygen saturation is 0% (that is, the absorption spectrum of reduced hemoglobin Hb). Furthermore, the short-dashed lines indicate absorption spectrums of hemoglobin (mixture of oxygenated hemoglobin and reduced hemoglobin) at oxygen saturations in between 0% and 100% (10, 20, 30, . . . , 90%).

As illustrated in FIG. 3, oxygenated hemoglobin and reduced hemoglobin have peak wavelengths differing from one another in the Q band. Specifically, oxygenated hemoglobin has an absorption peak P1 in the vicinity of the 542 nm wavelength and an absorption peak P3 in the vicinity of the 578 nm wavelength. On the other hand, reduced hemoglobin has an absorption peak P2 in the vicinity of the 558 nm wavelength. FIG. 3 shows a two-component absorption spectrum in which the sum of the concentrations of the components (oxygenated hemoglobin and reduced hemoglobin) is constant. Thus, isosbestic points E1, E2, E3, and E4 at which absorption is constant regardless of the concentrations of the respective components (that is, oxygen saturation) appear. In the following description, the wavelength region between the isosbestic points E1 and E2 is referred to as a “wavelength range R1”, the wavelength region between the isosbestic points E2 and E3 is referred to as a “wavelength range R2”, and the wavelength region between the isosbestic points E3 and E4 is referred to as a “wavelength range R3”. Furthermore, the wavelength region between the isosbestic points E1 and E4 (that is, the combination of the wavelength regions R1, R2, and R3) is referred to as a “wavelength range RO”.

As illustrated in FIG. 3, absorption increases or decreases monotonically relative to oxygen saturation between adjacent isosbestic points. Furthermore, absorption by hemoglobin changes substantially linearly relative to oxygen saturation between adjacent isosbestic points.

Specifically, absorption AR1 and AR3 by hemoglobin within the wavelength regions R1 and R3 increases monotonically and linearly relative to the concentration of oxygenated hemoglobin (oxygen saturation), and absorption AR2 by hemoglobin within the wavelength region R2 increases monotonically and linearly relative to the concentration of reduced hemoglobin (1-oxygen saturation).

The first special observation filter Fs1 is an optical bandpass filter that selectively transmits light of the 550 nm band (in other words, a bandpass filter having a first transmission band in the vicinity of the 550 nm wavelength). For example, the first transmission band is included within the wavelength band of 520 nm to 590 nm, and is 526 nm to 586 nm. As illustrated in FIG. 3, the first special observation filter Fs1 has a spectral characteristic of transmitting light within a wavelength range from the isosbestic point E1 to the isosbestic point E4 (that is, the wavelength range RO) with low loss and blocking light of other wavelength regions.

The second special observation filter Fs2 is also an optical bandpass filter that selectively transmits light of the 550 nm band (in other words, a bandpass filter having a second transmission band that is in the vicinity of the 550 nm wavelength and is narrower than the first transmission band). For example, the second transmission band is included within the wavelength band of 520 nm to 590 nm, and is 546 nm to 570 nm. As illustrated in FIG. 3, the second special observation filter Fs2 has a spectral characteristic of transmitting light within the wavelength range from the isosbestic point E2 to the isosbestic point E3 (that is, the wavelength range R2) with low loss and blocking light of other wavelength regions.

The normal observation filter Fn is an ultraviolet cut filter. The normal observation filter Fn may be replaced with a simple opening (without an optical filter) or a slit (without an optical filter) also achieving an aperture function.

The driver 263 drives the DC motor 262 under control of the system controller 202. The DC motor 262 causes the rotary turret 261 to rotate at a fixed speed when a drive current is supplied to the DC motor 262 from the driver 263.

When the rotary turret 261 is rotated by the DC motor 262, the rotary filter portion 260 sequentially inserts the optical filters, which are the normal observation filter Fn, the first special observation filter Fs1, and the second special observation filter Fs2, into the optical path of the irradiation light L at timings that are in synchronization with the imaging cycle (frame cycle). Due to this, irradiation light having different spectrums can be sequentially taken out from the irradiation light L emitted by the lamp 208, at timings that are in synchronization with the frame cycle. Note that in the following description, the term “frame” may be replaced with the term “field”. In the present embodiment, the frame cycle and the field cycle are 1/30 seconds and 1/60 seconds, respectively.

Here, for convenience of description, irradiation light L after passing through the first special observation filter Fs1 is referred to as “first special observation light Ls1”, irradiation light L after passing through the second special observation filter Fs2 is referred to as “second special observation light Ls2”, and irradiation light L after passing through the normal observation filter Fn is referred to as “normal light Ln”.

During the rotation, the rotary turret 261 cyclically takes out the normal light Ln, the first special observation light Ls1, and the second special observation light Ls2 from the normal observation filter Fn, the first special observation filter Fs1, and the second special observation filter Fs2, respectively.

Note that an opening (not shown) formed near the outer circumference of the rotary turret 261 is detected by using the photo-interrupter 264, whereby the rotational position and phase of the rotary turret 261 are controlled.

In accordance with the instructions from the operator input from the operation panel 214, the system controller 202 switches the observation mode of the electronic endoscope system 1. In the present embodiment, the observation modes that can be switched and set include a normal observation mode and a special observation mode.

### Normal Observation Mode

The following describes the operations of the electronic endoscope system 1 during the normal observation mode.

During the normal observation mode, the system controller 202 controls the driver 263 and thereby stops the rotary turret 261 at a position at which the normal observation filter Fn is inserted into the optical path. Due to this, the irradiation light L is filtered by the normal observation filter Fn into the normal light Ln. The light amount of the normal light Ln is regulated to an appropriate light amount through diaphragm blades (not shown), and the normal light Ln is condensed by the light condenser lens 210 onto an incident end surface of a light-carrying bundle (LCB) 102 and is made to enter the LCB 102. Note that, during the normal observation mode, the system controller 202 may retract the rotary turret 261 to a position withdrawn from the optical path, in place of stopping the rotary turret 261 at the position at which the normal observation filter Fn is inserted into the optical path.

The normal light Ln that entered the LCB 102 propagates through the LCB 102. The normal light Ln that propagated through the LCB 102 is emitted from an emission end surface of the LCB 102, which is arranged at the leading end of the electronic scope 100, whereby biological tissue is irradiated with the normal light Ln passing through a light distribution lens 104. Returning light from the biological tissue irradiated with the normal light Ln from the light distribution lens 104 passes through an objective lens 106 and forms an optical image on a light-receiving surface of a solid-state imaging element 108.

The solid-state imaging element 108 is a single-chip color charged coupled device (CCD) image sensor having a Bayer-type pixel arrangement. The solid-state imaging element 108 accumulates the optical image formed on each pixel on the light-receiving surface in the form of electric charge in accordance with the light amount, and generates and outputs image signals of red (R), green (G), and blue (B). Note that the solid-state imaging element 108 is not limited to a CCD image sensor, and may be replaced with a complementary metal oxide semiconductor (CMOS) image sensor or other types of imaging devices. Furthermore, the solid-state imaging element 108 may be that on which complementary color filters are mounted.

A driver signal processing circuit 110 is provided inside a connection portion of the electronic scope 100. Image signals of the biological tissue irradiated with the light from the light distribution lens 104 are input from the solid-state imaging element 108 to the driver signal processing circuit 110 at a frame cycle. The driver signal processing circuit 110 performs predetermined processing on the image signals input from the solid-state imaging element 108 and outputs the image signals to a signal processing circuit 220 of the processor 200.

Furthermore, the driver signal processing circuit 110 also accesses a memory 112 and reads out specific information of the electronic scope 100. The specific information of the electronic scope 100 stored in the memory 112 includes, for example, the number of pixels, sensitivity, operable frame rates, model number, etc., of the solid-state imaging element 108. The driver signal processing circuit 110 outputs the specific information read out from the memory 112 to the system controller 202.

The system controller 202 performs various types of calculation based on the specific information of the electronic scope 100 to generate a control signal. The system controller 202 uses the generated control signal to control timing and operations of various circuits inside the processor 200 so that processing suitable for the electronic scope connected to the processor 200 is performed.

In accordance with timing control by the system controller 202, the timing controller 204 supplies a clock pulse to the driver signal processing circuit 110. In accordance with the clock pulse supplied from the timing controller 204, the driver signal processing circuit 110 drives and controls the solid-state imaging element 108 at timings that are in synchronization with the frame rate of images processed at the processor 200 side.

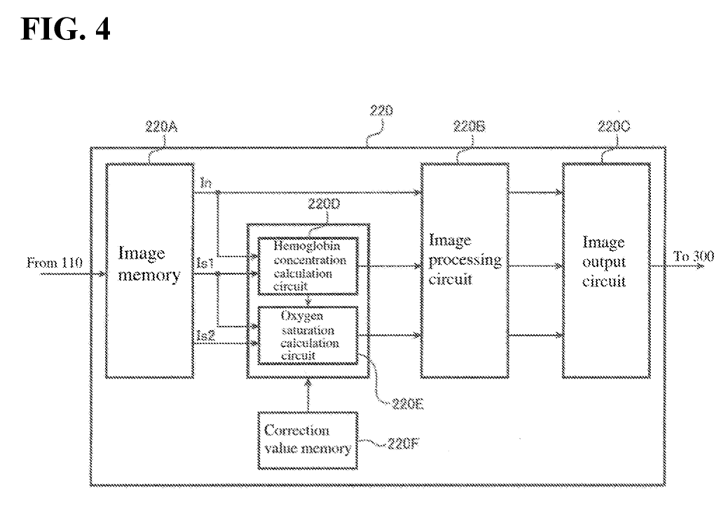

FIG. 4 is a block diagram illustrating a configuration of the signal processing circuit 220. As illustrated in FIG. 4, the signal processing circuit 220 has an image memory 220A, an image processing circuit 220B, an image output circuit 220C, a hemoglobin concentration calculation circuit 220D, an oxygen saturation calculation circuit 220E, and a correction value memory 220F.

The image memory 220A buffers the image signal input from the driver signal processing circuit 110 in units of one 1 frame cycle and outputs the image signal to the image processing circuit 220B in accordance with timing control by the timing controller 204.

The image processing circuit 220B performs predetermined signal processing with respect to the image signal input from the image memory 220A and outputs the image signal to the image output circuit 220C. The predetermined signal processing includes de-mosaic processing, a matrix operation, Y/C separation, etc.

The image output circuit 220C processes the image signal input from the image processing circuit 220B to generate screen data for monitor display, and converts the generated screen data for monitor display into a predetermined video format signal. The video format signal yielded by the conversion is output to the monitor 300. Due to this, a normal color image of the biological tissue is displayed on the display screen of the monitor 300.

### Special Observation Mode

The following describes the operations of the electronic endoscope system 1 during the special observation mode.

During the special observation mode, the system controller 202 controls the driver 263 and thereby controls the rotary turret 261 to rotate at a fixed speed and sequentially inserts the optical filters, which are the normal observation filter Fn, the first special observation filter Fs1, and the second special observation filter Fs2, into the optical path of the irradiation light L at timings that are in synchronization with the imaging cycle (frame cycle). Due to this, during the rotation of the rotary turret 261, the normal light Ln, the first special observation light Ls1, and the second special observation light Ls2 are taken out from the incident irradiation light L from the lamp 208 by the normal observation filter Fn, the first special observation filter Fs1, and the second special observation filter Fs2, respectively. Due to this, the biological tissue is sequentially irradiated with irradiation light, or that is, the normal light Ln, the first special observation light Ls1, and the second special observation light Ls2 at timings that are in synchronization with the frame cycle.

The solid-state imaging element 108 takes images of the biological tissue sequentially irradiated with each type of irradiation light (the normal light Ln, the first special observation light Ls1, and the second special observation light Ls2), and outputs the image signals to the driver signal processing circuit 110. In the following, for convenience of description, an image signal of the biological tissue taken during the period of irradiation with the normal light Ln is referred to as a “normal image signal In”, an image signal of the biological tissue taken during the period of irradiation with the first special observation light Ls1 is referred to as a “first special image signal Is1”, and an image signal of the biological tissue taken during the period of irradiation with the second special observation light Ls2 is referred to as a “second special image signal Is2”.

The image memory 220A buffers the normal image signal In, the first special image signal Is1, and the second special image signal Is2 that are sequentially input thereto and outputs the image signals to the image processing circuit 220B, the hemoglobin concentration calculation circuit 220D, and the oxygen saturation calculation circuit 220E in accordance with timing control by the timing controller 204.

In more detail, the image memory 220A outputs the normal image signal In (all of the R, G, and B signals) to the image processing circuit 220B, outputs the normal image signal In (only the R signal) and the first special image signal Is1 to the hemoglobin concentration calculation circuit 220D, and outputs the first special image signal Is1 and the second special image signal Is2 to the oxygen saturation calculation circuit 220E.

The normal image signal In input to the image processing circuit 220B is subjected to predetermined signal processing and is output to the image output circuit 220C, similarly to during the normal observation mode.

When the electronic endoscope system 1 has an individual difference (for example, an individual difference in terms of the spectral characteristic of the first special observation filter Fs1, the sensitivity of the solid-state imaging element 108, etc.), the result of the calculation of hemoglobin concentration by the hemoglobin concentration calculation circuit 220D will include an error. The spectral characteristic of the detection light (the first special observation light Ls1) for detecting the hemoglobin amount in biological tissue has a dominant influence over the hemoglobin concentration calculation error.

Thus, in the present embodiment, a first special image signal Is1 and a normal image signal In (only the R signal) that serve as references are acquired at a timing such as shipping from a factory, by taking images of a reference subject with uniform reflectance, such as a gray card, a white board, or the like. Here, the first special image signal Is1 and the normal image signal In (only the R signal) serving as references are denoted as Is1₀ and In₀, respectively. Subsequently, a correction value γ (=α/α₀) (a first correction value), which is for correcting a luminance signal ratio between the first special image signal Is1 and the normal image signal In (only the R signal) that have been acquired, or that is, the ratio Is1₀/In₀ (a ratio α₀ between the luminance values of these image signals) into a designed (that is, an appropriate) ratio α, is calculated and stored to the correction value memory 220F. That is, the correction value γ can be expressed as follows: γ=α/α₀=α/(Is1₀/In₀).

Note that the first special image signal Is1 and the normal image signal In (only the R signal) that serve as references may be signals acquired by using a gray card or the like upon setting of white balance. In this case, the first correction value γ is calculated immediately after the white balance is set, and is stored to the correction value memory 220F.

The hemoglobin concentration calculation circuit 220D reads out the first correction value γ from the correction value memory 220F and corrects the normal image signal In (only the R signal) by using the first correction value γ that was read out. For example, the hemoglobin concentration calculation circuit 220D performs correction of In into In/γ. The hemoglobin concentration calculation circuit 220D divides the first special image signal Is1 by the corrected normal image signal In (only the R signal), or that is, calculates Is1/(In/γ)=Is1/In·γ (=Is1/In·(α·(In₀/Is1₀))) for example, and thereby acquires hemoglobin concentration information in which an error deriving from an individual difference of the electronic endoscope system 1 has been corrected.

In addition, in the present embodiment, the first special image signal Is1 is divided by the normal image signal In (only the R signal), which has a wavelength range for which absorption by hemoglobin inside biological tissue is low, whereby hemoglobin concentration information is acquired in which reflectance fluctuation due to the surface state of the biological tissue and the difference in irradiation light incident angle relative to the biological tissue is corrected. By acquiring the hemoglobin concentration information based on a ratio between the first special image signal Is1 and the normal image signal In (only the R signal) in such a manner, hemoglobin concentration information can be acquired in which not only the individual difference of the electronic endoscope system 1 but also reflectance fluctuation due to the surface state of the biological tissue and the difference in irradiation light incident angle relative to the biological tissue is suppressed.

The hemoglobin concentration calculation circuit 220D outputs the calculated hemoglobin concentration information to the image processing circuit 220B. Based on the hemoglobin concentration information input from the hemoglobin concentration calculation circuit 220D, the image processing circuit 220B generates a color map image (spectral image) in which an error deriving from the individual difference of the electronic endoscope system 1 is corrected. To provide an example, the image processing circuit 220B holds a reference table in which hemoglobin concentration values and predetermined display colors are associated, and generates an image signal (for convenience of description, referred to in the following as a “hemoglobin concentration image signal”) for a color map by allocating a display color in accordance with hemoglobin concentration to each pixel. The image processing circuit 220B outputs the generated hemoglobin concentration image signal to the image output circuit 220C.

Furthermore, when the electronic endoscope system 1 has an individual difference (for example, an individual difference in terms of the spectral characteristics of the first special image signal Is1 and the second special image signal Is2, the sensitivity of the solid-state imaging element 108, etc.), the result of the calculation of oxygen saturation by the oxygen saturation calculation circuit 220E will also include an error. The spectral characteristics of detection light (the first special image signal Is1 and the second special image signal Is2) for detecting the oxygen saturation in biological tissue have a dominant influence over the oxygen saturation calculation error.

Thus, in the present embodiment, in addition to the first special image signal Is1 and the normal image signal In (only the R signal) that serve as references, a second special image signal Is2 that serves as a reference is acquired at the timing such as shipping from a factory, by taking an image of the reference subject with uniform reflectance, such as a gray card, a white board, or the like. Here, the first special image signal Is1 and the second special image signal Is2 serving as references are denoted as Is1₀ and Is2₀, respectively. Subsequently, a correction value δ (=β/β₀) (a second correction value), which is for correcting a luminance signal ratio between the first special image signal Is1 and the second special image signal Is2 that have been acquired, or that is, the ratio Is1₀/Is2₀ (a ratio β₀ between the luminance values of these image signals) into a designed (that is, an appropriate) ratio β, is calculated and stored to the correction value memory 220F. That is, the correction value δ can be expressed as follows: δ=β/β₀=β/(Is1₀/Is2₀).

Note that the second special image signal Is2 that serves as a reference may be a signal acquired by using a gray card or the like upon setting of white balance. In this case, the second correction value is calculated immediately after the white balance is set, and is stored to the correction value memory 220F.

The oxygen saturation calculation circuit 220E reads out the first correction value γ from the correction value memory 220F and corrects the normal image signal In (only the R signal) by using the first correction value γ that was read out, or that is, calculates In/γ, and also reads out the second correction value δ from the correction value memory 220F and corrects the second special image signal Is2 by using the second correction value δ that was read out, or that is, calculates Is2/δ. The oxygen saturation calculation circuit 220E subtracts the corrected second special image signal Is2 (=Is2/δ) from the first special image signal Is1, and divides the value yielded through the subtraction by the corrected normal image signal In (only the R signal) (=In/γ). Thus, oxygen saturation information in which an error deriving from an individual difference of the electronic endoscope system 1 has been corrected can be yielded.

In addition, in the present embodiment, the above-described value yielded through subtraction is divided by the normal image signal In (only the R signal), which has a wavelength range for which absorption by hemoglobin inside the biological tissue is low, thus obtaining oxygen saturation information in which reflectance fluctuation due to the surface state of the biological tissue and the difference in irradiation light incident angle relative to the biological tissue is corrected. By acquiring the oxygen saturation information based on a ratio between the first special image signal Is1 and the second special image signal Is2 in such a manner, oxygen saturation information can be acquired in which not only the individual difference of the electronic endoscope system 1 but also reflectance fluctuation due to the surface state of the biological tissue and the difference in irradiation light incident angle relative to the biological tissue is suppressed.

The oxygen saturation calculation circuit 220E outputs the calculated oxygen saturation information to the image processing circuit 220B. Based on the oxygen saturation information input from the oxygen saturation calculation circuit 220E, the image processing circuit 220B generates a color map image (spectral image) in which an error deriving from the individual difference of the electronic endoscope system 1 is corrected. To provide an example, the image processing circuit 220B holds a reference table in which oxygen saturation values and predetermined display colors are associated, and generates an image signal (for convenience of description, referred to in the following as an “oxygen saturation image signal”) for a color map by allocating a display color in accordance with oxygen saturation to each pixel. The image processing circuit 220B outputs the generated oxygen saturation image signal to the image output circuit 220C.

The image output circuit 220C processes the normal image signal In input from the image processing circuit 220B to generate a normal color image of the biological tissue. Also, the image output circuit 220C processes the hemoglobin concentration image signal input from the hemoglobin concentration calculation circuit 220D and the oxygen saturation image signal input from the oxygen saturation calculation circuit 220E to generate a hemoglobin concentration color map image and an oxygen saturation color map image, respectively.

The operator can operate the operation panel 214 and thereby set the display state of observation images during the special observation mode. The image output circuit 220C uses the generated images to generate screen data for monitor display that is in accordance with the display state that is set, and converts the generated screen data for monitor display into a predetermined video format signal. The video format signal yielded by the conversion is output to the monitor 300. Thus, an image in accordance with the set display state is displayed.

To provide an example, the following states (1) through (5) can be given as display states of observation images that can be set during the special observation mode.

(1) A state in which an image of one system, among the images of three systems (the normal color image of the biological tissue, the hemoglobin concentration color map image, and the oxygen saturation color map image) is displayed.
(2) A state in which the images of the three systems or images of two of the three systems are displayed side by side in one screen with the same size.
(3) A state in which an image of one system is displayed in large screen display and the images of the two remaining systems or an image of one of the two systems are/is displayed in small screen display.
(4) A state in which images of two of the three systems are displayed in an overlaid state.
(5) A state in which images of all three systems are displayed in an overlaid state.

As described above, the correction values γ and δ are values set so that the ratios α₀ and β₀ between luminance values of specific pairs among, for example, the normal image signal In, the first special image signal Is1, and the second special image signal Is2, which are yielded when images of the reference subject irradiated with multiple types of irradiation light are taken, equal the predetermined target ratios α and β. Thus, the correction performed in the present embodiment is useful in that, when the hemoglobin concentration information and the oxygen saturation information are calculated by using ratios between image signals of at least two different systems, it is possible to perform correction that is not dependent on the signal levels of the normal image signal In, the first special image signal Is1, the second special image signal Is2.

Furthermore, since the image signals of specific systems that are yielded from the reference subject and are used in the calculation of the correction values are luminance values, accurate correction becomes possible even in the case of multiple types of irradiation light having different wavelength bands and also different light intensities.

Furthermore, the filters used in the above-described embodiment include the first special observation filter Fs1 having the first transmission band, which is included within the wavelength band of 520 nm to 590 nm, and the second special observation filter Fs2 having the second transmission band, which is included within the wavelength band of 520 nm to 590 nm and is narrower than the first transmission band. Accordingly, irradiation light that includes, as a wavelength band, a band for which hemoglobin indicates strong absorption can be emitted. Due to this, hemoglobin concentration and oxygen saturation can be calculated accurately.

Furthermore, in the above-described embodiment, image signals of the subject are corrected by using the correction values γ and δ, which are set so that the ratios α₀ and β₀ between luminance values of reference image signals of at least two systems that are yielded when images are taken of the reference subject irradiated with multiple types of irradiation light, such as the normal light Ln, the first special observation light Ls1, and the second special observation light Ls2, equal the predetermined target ratios α and β. Accordingly, it is possible to suppress degradation in accuracy (variation), deriving from an individual difference of the system, of feature amount information calculated by using ratios between image signals, such as hemoglobin concentration and oxygen saturation. Thus, the feature amounts to be calculated are preferably amounts determinable based on ratios between luminance values of image signals of at least two systems.

Note that, in order to acquire the oxygen saturation information accurately, it is preferable that the wavelength band of one of the multiple types of irradiation light is demarcated from the wavelength band of another type of irradiation light by an isosbestic point corresponding to a switch between levels of the spectral waveform of light absorbance of oxygenated hemoglobin and the spectral waveform of light absorbance of reduced hemoglobin. In the example illustrated in FIG. 3, the wavelength band of the second special observation light Ls2 is demarcated from the wavelength band of the first special observation light Ls1 at the isosbestic points E2 and E3.

Furthermore, in order to acquire the oxygen saturation information accurately, it is preferable that the wavelength band of one of the multiple types of irradiation light is included within a wavelength band between isosbestic points that are adjacent in the wavelength direction, among a plurality of isosbestic points corresponding to a switch between levels of the spectral waveform of the light absorbance of oxygenated hemoglobin and the spectral waveform of light absorbance of reduced hemoglobin. In the example illustrated in FIG. 3, the second special observation light Ls2 is included within the wavelength band between the isosbestic points E2 and E3.

The wavelength band of such irradiation light is not limited to the range of 500 nm to 600 nm illustrated in FIG. 3. For example, application is also possible to a wavelength band in which light absorbance changes around isosbestic points depending upon hemoglobin oxygen saturation. For example, a certain wavelength band on the long-wavelength side or the short-wavelength side of one of the isosbestic points in the wavelength band from 400 nm to 500 nm can be set as the wavelength band of the irradiation light.

In the above, description has been provided of an exemplary embodiment of the present disclosure. Embodiments of the present disclosure are not limited to what is described above, and various modifications are possible within the scope of the technical concept of the present disclosure. For example, matters yielded by combining, as appropriate, an embodiment explicitly disclosed as an example in the description, etc., or obvious embodiments, etc., are also included among embodiments of the present application.

In the above-described embodiment, the light source device is built into the processor 200. However, in another embodiment, a configuration may be adopted in which the processor 200 and the light source device are separate. In this case, a wired or wireless communication means for the transmission/reception of timing signals between the processor 200 and the light source device will be provided.

Furthermore, in the above-described embodiment, the normal observation filter Fn, the first special observation filter Fs1, and the second special observation filter Fs2 are arranged in the rotary turret 261. However, in another embodiment, optical filters having different spectral characteristics, such as an infrared light observation filter, a fluorescence observation filter, etc., may be arranged in the rotary turret 261.

Furthermore, in the above-described embodiment, a configuration is adopted in which the rotary filter portion 260 is provided for the lamp 208 and filtering is performed with respect to the irradiation light L. However, the present disclosure is not limited to this configuration. For example, a configuration may be adopted in which the rotary filter portion 260 is provided for the solid-state imaging element 108 and filtering is performed with respect to returning light from the subject.

### REFERENCE SIGNS LIST

1 Electronic endoscope system
100 Electronic scope
102 LCB
104 Light distribution lens
106 Objective lens
108 Solid-state imaging element
110 Driver signal processing circuit
112 Memory
200 Processor
202 System controller
204 Timing controller
206 Lamp light source ignitor
208 Lamp
210 Condenser lens
212 Memory
214 Operation panel
220 Signal processing circuit
220A Image memory
220B Image processing circuit
220C Image output circuit
220D Hemoglobin concentration calculation circuit
220E Oxygen saturation calculation circuit
220F Correction value memory
260 Rotary filter portion
261 Rotary turret
Fn Normal observation filter
Fs1 First special observation filter
Fs2 Second special observation filter
262 DC motor
263 Driver
264 Photo-interrupter

1. An electronic endoscope system comprising:
an irradiation means for sequentially irradiating a subject with a plurality of types of irradiation light having different spectrums;
an image signal generation means for sequentially taking images of the subject sequentially irradiated with the plurality of types of irradiation light, and generating image signals of the subject irradiated with the respective types of irradiation light as image signals of a plurality of systems;
a storage means having a predetermined correction value stored therein in advance; and
a spectral image generation means for generating a spectral image indicating a distribution of a feature amount of the subject, the feature amount being determinable based on image signals of at least two systems among the image signals of the plurality of systems, wherein
the spectral image generation means includes means for correcting an image signal of at least one system among the image signals of the at least two systems, when generating the spectral image based on the image signals of the at least two systems, based on the correction value stored in advance in the storage means.

2. The electronic endoscope system according to claim 1, wherein
the correction value is a value calculated in advance based on a ratio between luminance values of a specific pair of image signals among the image signals of the at least two systems, and
the spectral image generation means includes means for correcting one image signal among the specific pair of image signals, when generating the spectral image based on the image signals of the at least two systems, based on the correction value.

3. The electronic endoscope system according to claim 2, wherein
the correction value is a correction value set so that a ratio between luminance values of the specific pair of image signals equals a predetermined target ratio, the luminance values of the specific pair of image signals being yielded when images are taken of a reference subject irradiated with the plurality of types of irradiation light.

4. The electronic endoscope system according to claim 1, wherein
the irradiation means includes:
a light source for emitting light;
a rotary member in which a plurality of light transmission regions having different transmission bands are arranged side by side in a circumferential direction;
a means for causing the rotary member to rotate and sequentially inserting the plurality of light transmission regions into an optical path of the light in order to sequentially take out the plurality of types of irradiation light having different spectrums from the light; and
a means for sequentially emitting, toward the subject, the plurality of types of irradiation light that are sequentially taken out.

5. The electronic endoscope system according to claim 4, wherein
the plurality of light transmission regions are optical filters that are arranged in the rotary member, the optical filters including:
a first filter having a first transmission band, the first transmission band being included within a wavelength band of 520 nm to 590 nm;
a second filter having a second transmission band, the second transmission band being included within the wavelength band of 520 nm to 590 nm and being narrower than the first transmission band; and
a filter that transmits white light.

6. The electronic endoscope system according to claim 5, wherein
the correction value includes a first correction value, the first correction value being a value for correcting, into a predetermined first ratio, a ratio between a luminance value of an image signal constituted of a part of a plurality of components constituting an image signal of the reference subject irradiated with the white light and a luminance value of an image signal of the reference subject irradiated with light filtered by the first filter, and
the spectral image generation means includes
means for correcting an image signal A constituted of a part of a plurality of components constituting an image signal of the subject irradiated with the white light based on the first correction value,
means for dividing an image signal B of the subject irradiated with light filtered by the first filter by the image signal A corrected with the first correction value to acquire hemoglobin concentration information of the subject, and
means for generating a spectral image indicating hemoglobin concentration based on the acquired hemoglobin concentration information.

7. The electronic endoscope system according to claim 6, wherein
the correction value includes a second correction value, the second correction value being a value for correcting, into a predetermined second ratio, a ratio between the luminance value of the image signal of the reference subject irradiated with the light filtered by the first filter and a luminance value of an image signal of the reference subject irradiated with light filtered by the second filter, and
the spectral image generation means includes
means for correcting the image signal A constituted of a part of the plurality of components constituting the image signal of the subject irradiated with the white light based on the first correction value, and also corrects an image signal C of the subject irradiated with light filtered by the second filter based on the second correction value,
means for subtracting the image signal C corrected using the second correction value from the image signal B of the subject irradiated with the light filtered by the first filter,
means for dividing the value after the subtraction by the image signal A corrected using the first correction value to acquire oxygen saturation information of the subject, and
means for generating a spectral image indicating oxygen saturation based on the acquired oxygen saturation information.

8. An electronic endoscope system comprising:
an irradiation means for sequentially irradiating a subject with a plurality of types of irradiation light having different spectrums;
an image signal generation means for sequentially taking images of the subject sequentially irradiated with the plurality of types of irradiation light, and generating image signals of the subject irradiated with the respective types of irradiation light as image signals of a plurality of systems; and
a spectral image generation means for generating a spectral image indicating a distribution of a feature amount of the subject, the feature amount being determinable based on image signals of at least two systems among the image signals of the plurality of systems, wherein
the spectral image generation means includes means for calculating the feature amount by correcting one of the image signals of the at least two systems based on a predetermined correction value, and
the predetermined correction value is a correction value set so that a ratio between luminance values of reference image signals of the at least two systems equals a predetermined target ratio, the reference image signals of the at least two systems being yielded when images are taken of a reference subject irradiated with the irradiation light.

9. The electronic endoscope system according to claim 8, wherein
the feature amount is an amount determinable based on a ratio between luminance values of the image signals of the at least two systems.

10. The electronic endoscope system according to claim 1, wherein
a wavelength band of one type of irradiation light, among the plurality of types of irradiation light, is demarcated from a wavelength band of another type of irradiation light, among the multiple types of irradiation light, by an isosbestic point corresponding to a switch between levels of a spectral waveform of light absorbance of oxygenated hemoglobin and a spectral waveform of light absorbance of reduced hemoglobin.

11. The electronic endoscope system according to claim 1₀, wherein
a wavelength band of one type of irradiation light, among the multiple types of irradiation light, is included within a wavelength band between isosbestic points that are adjacent in a wavelength direction, among a plurality of isosbestic points corresponding to a switch between levels of the spectral waveform of light absorbance of oxygenated hemoglobin and the spectral waveform of light absorbance of reduced hemoglobin.

12. The electronic endoscope system according to claim 8, wherein
a wavelength band of one type of irradiation light, among the plurality of types of irradiation light, is demarcated from a wavelength band of another type of irradiation light, among the multiple types of irradiation light, by an isosbestic point corresponding to a switch between levels of a spectral waveform of light absorbance of oxygenated hemoglobin and a spectral waveform of light absorbance of reduced hemoglobin.

13. The electronic endoscope system according to claim 12, wherein
a wavelength band of one type of irradiation light, among the multiple types of irradiation light, is included within a wavelength band between isosbestic points that are adjacent in a wavelength direction, among a plurality of isosbestic points corresponding to a switch between levels of the spectral waveform of light absorbance of oxygenated hemoglobin and the spectral waveform of light absorbance of reduced hemoglobin.

14. An electronic endoscope system comprising:
an irradiation assembly for sequentially irradiating a subject with a plurality of types of irradiation light having different spectrums;
a driver signal processing circuit configured to sequentially take images of the subject sequentially irradiated with the plurality of types of irradiation light, and to generate image signals of the subject irradiated with the respective types of irradiation light as image signals of a plurality of systems;
a memory having a predetermined correction value stored therein in advance;
a calculation circuit configured to correct an image signal of at least one system among the image signals of the at least two systems, based on the correction value stored in advance in the memory, to produce a corrected image signal; and
an image processing circuit configured to generate a spectral image indicating a distribution of a feature amount of the subject, the feature amount being determinable based on image signals, including the corrected image signal, of at least two systems among the image signals of the plurality of systems.

15. The electronic endoscope system according to claim 14, wherein
the correction value is a value calculated in advance based on a ratio between luminance values of a specific pair of image signals among the image signals of the at least two systems, and
the calculation circuit is configured to correct one image signal among the specific pair of image signals to produce the corrected image signal.

16. The electronic endoscope system according to claim 15, wherein
the correction value is a correction value set so that a ratio between luminance values of the specific pair of image signals equals a predetermined target ratio, the luminance values of the specific pair of image signals being yielded when images are taken of a reference subject irradiated with the plurality of types of irradiation light.

17. The electronic endoscope system according to claim 14, wherein
the irradiation assembly includes:
a light source configured to emit light;
a rotary member in which a plurality of light transmission regions having different transmission bands are arranged side by side in a circumferential direction;
a motor arranged to cause the rotary member to rotate and to sequentially insert the plurality of light transmission regions into an optical path of the light in order to sequentially take out the plurality of types of irradiation light having different spectrums from the light; and
a light carrying bundle arranged to sequentially emit, toward the subject, the plurality of types of irradiation light that are sequentially taken out.

18. The electronic endoscope system according to claim 17, wherein
the feature amount is an amount determinable based on a ratio between luminance values of the image signals of the at least two systems.

19. The electronic endoscope system according to claim 14, wherein
a wavelength band of one type of irradiation light, among the plurality of types of irradiation light, is demarcated from a wavelength band of another type of irradiation light, among the multiple types of irradiation light, by an isosbestic point corresponding to a switch between levels of a spectral waveform of light absorbance of oxygenated hemoglobin and a spectral waveform of light absorbance of reduced hemoglobin.

20. The electronic endoscope system according to claim 19, wherein
a wavelength band of one type of irradiation light, among the multiple types of irradiation light, is included within a wavelength band between isosbestic points that are adjacent in a wavelength direction, among a plurality of isosbestic points corresponding to a switch between levels of the spectral waveform of light absorbance of oxygenated hemoglobin and the spectral waveform of light absorbance of reduced hemoglobin.
